(19) United States
(12) Patent Application Publication
(10) Pub. No.: US 2024/0199689 A1
(43) Pub. Date: Jun. 20, 2024

(54) METHOD FOR THE ANALYSIS OF RELATED SUBSTANCES IN GERMINAL PEPTIDE PREPARATION

(71) Applicant: ZHAOKE (GUANGZHOU) OPHTHALMOLOGY PHARMACEUTICAL LIMITED, Guangzhou (CN)

(72) Inventors: Zhijun XIE, Guangzhou (CN); Ping TAN, Guangzhou (CN)

(73) Assignee: ZHAOKE (GUANGZHOU) OPHTHALMOLOGY PHARMACEUTICAL LIMITED, Guangzhou (CN)

(21) Appl. No.: 18/591,010

(22) Filed: Feb. 29, 2024

Related U.S. Application Data
(63) Continuation of application No. PCT/CN2023/093879, filed on May 12, 2023.

(30) Foreign Application Priority Data
Aug. 2, 2022 (CN) 202210921653.8

(51) Int. Cl. C07K 1/16

(52) U.S. Cl. CPC C07K 1/16

(57) ABSTRACT

A method for analysis of related substances in a germinal peptide preparation is provided. The method refers to high-performance liquid chromatography (HPLC), and the HPLC is conducted under the following chromatographic conditions: a chromatographic column: a YMC-Triart C18 chromatographic column, 4.6×150 mm, 3 μm; a column temperature: 35° C.; an injection volume: 100 μL; a detection wavelength: 210 nm; a mobile phase A: a 0.1% trifluoroacetic acid (TFA) aqueous solution; a mobile phase B: 0.1% TFA-acetonitrile; and gradient elution as follows: 0 min: a volume ratio of the mobile phase A to the mobile phase B is 100:0; 8 min to 13 min: the volume ratio of the mobile phase A to the mobile phase B is 85:15; and 13 min to 20 min: the volume ratio of the mobile phase A to the mobile phase B is 100:0.

### CROSS REFERENCE TO THE RELATED APPLICATIONS

This application is a continuation application of the national phase entry of International Application No. PCT/CN2023/093879, filed on May 12, 2023, which is based upon and claims priority to Chinese Patent Application No. 202210921653.8, filed on Aug. 2, 2022, the entire contents of which are incorporated herein by reference.

### TECHNICAL FIELD

The present disclosure relates to the field of analytical chemistry, and in particular relates to a method for analysis of related substances in a germinal peptide preparation.

### BACKGROUND

Germinal peptide is a core functional region in thymosin beta 4 (Tβ4) to bind to actin. It has been found by scientists from the National Institutes of Health (NIH) in the United States that germinal peptide is composed of 7 amino acids and has an amino acid sequence of leucine-lysine-lysine-threonine-glutamic acid-threonine-glutamine (L-K-K-T-E-T-Q). Studies have shown that germinal peptide has various functions, such as binding to actin, promoting hair growth, stimulating angiogenesis, accelerating epidermal wound healing, and promoting cell migration.

Results of pharmacodynamic and safety evaluation tests show that germinal peptide is safe and non-toxic and has the effects of promoting corneal epithelial repair and reducing inflammatory responses in rats. Indications of germinal peptide eye drops are corneal epithelial punctate defects caused by dry eye disease and corneal epithelial patchy defects caused by various factors (including I and II-degree burns, mechanical injuries, and iatrogenic factors).

During the synthesis of germinal peptide, epimers, deleted peptides, inserted peptides, or the like may be produced, including 7 epimers of all individual sites (among which a diastereomer of Thr is a D-allo structure), and 6 deleted peptide impurities, 13 inserted peptide impurities, 7 misconnected peptide impurities, and 10 broken peptide impurities that are easily produced. IM020/Glu7 impurities easily produced due to degradation are synthesized according to their sequence structures and processes, and a total of 44 impurities are investigated.

There is currently no detection method for an active pharmaceutical ingredient (API) in the Chinese Pharmacopoeia, and a detection method for API provided by Zhejiang Peptites Biotech Co., Ltd. (a germinal peptide manufacturer) is as follows:
a chromatographic column: YMC-PackODS-AQ 4.6×250 mm, 3 μm (C18 high-performance chromatographic column manufactured by YMC, Japan);
a column temperature: 45° C.;
a mobile phase A: 50 mM phosphate-buffered saline (pH 6.0); a mobile phase B: 50% methanol;
a flow rate: 0.8 mL/min;
an injection volume: 20 μL (2 mg/mL);
a detection wavelength: ultraviolet (UV) 210 nm; and
an elution gradient:

| Time (min) | Mobile phase A (%) | Mobile phase B (%) |
|---|---|---|
| 0 | 98 | 2 |
| 30 | 92 | 8 |
| 40 | 40 | 60 |
| 50 | 40 | 60 |
| 51 | 98 | 2 |
| 60 | 98 | 2 |

As shown in FIGS. 1-3, FIG. 1 is a spectrum of germinal peptide detection in the prior art, FIG. 2 is a spectrum of API and an impurity A (Glu⁷-germinal peptide) in a germinal peptide in the prior art, and FIG. 3 is a spectrum of API, an impurity B (D-Leu¹-germinal peptide), and an impurity C (Di-Glu⁵-germinal peptide) in a germinal peptide in the prior art. The above detection method has the following problems: The detection time is as long as 50 minutes or more, which affects a detection efficiency. There is poor specificity for simultaneous detection of a plurality of impurities, and there is poor separation of impurity peaks from a main peak. Moreover, after mixed standard detection, there is very poor separation between the first two impurities (far less than 1.5), and impurity peaks behind a main peak cannot reach baseline separation. Thus, this detection method cannot allow the simultaneous detection of a plurality of known impurities. Therefore, a novel purity detection method is required to detect and separate the above various known impurities and other unknown impurities.

Therefore, it is necessary to develop an analysis and detection method that can effectively separate related substances in a germinal peptide preparation and eliminate the interference by adjuvants.

### SUMMARY

For impurities in a germinal peptide preparation, the inventors of the present disclosure develop a plurality of chromatographic conditions for separation, and finally determine a process capable of fully controlling these impurities through process research. In the present disclosure, a method is optimized for detection of some common impurities.

In a first aspect, the present disclosure provides a method for efficient analysis of related substances in a germinal peptide preparation, which greatly shortens an analysis time. The method includes the following steps: preparing a sample; and testing the sample by high-performance liquid chromatography (HPLC), where a chromatographic column with octadecylsilane-bonded silica gel as a packing is adopted for the HPLC.

Further, the method can effectively separate germinal peptide from related substances, and preferably, the related substances refer to Glu⁷-germinal peptide (impurity A), D-Leu¹-germinal peptide (impurity B), and Di-Glu⁵-germinal peptide (impurity C).

Further, mobile phases for the HPLC include a mobile phase A and a mobile phase B. The mobile phase A is a trifluoroacetic acid (TFA) aqueous solution in which a volume ratio of the TFA to water is 0.05:100 to 0.15:100, such as 0.06:100, 0.07:100, 0.08:100, 0.09:100, 0.10:100, 0.11:100, 0.12:100, 0.13:100, or 0.14:100. Preferably, the volume ratio of the TFA to the water in the mobile phase A is 0.1:100.

Further, the mobile phase B is TFA-acetonitrile in which a volume ratio of the TFA to the acetonitrile is 0.05:100 to 0.15:100, such as 0.06:100, 0.07:100, 0.08:100, 0.09:100, 0.10:100, 0.11:100, 0.12:100, 0.13:100, or 0.14:100. Preferably, the volume ratio of the TFA to the acetonitrile in the mobile phase B is 0.1:100.

Further, a detection wavelength for the HPLC is 200 nm to 220 nm, such as 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, or 219 nm. Preferably, the detection wavelength is 210 nm.

Further, a column temperature of the chromatographic column is 30° C. to 40° C., such as 31° C., 32° C., 33° C., 34° C., 35° C., 36° C., 37° C., 38° C., or 39° C. Preferably, the column temperature of the chromatographic column is 35° C.

Further, an injection volume for the HPLC is 90 μL to 110 μL, such as 91 μL, 92 μL, 93 μL, 94 μL, 95 μL, 96 μL, 97 μL, 98 μL, 99 μL, 100 μL, 101 μL, 102 μL, 103 μL, 104 μL, 105 μL, 106 μL, 107 μL, 108 μL, or 109 μL. Preferably, the injection volume is 100 μL.

Further, a flow rate for the HPLC is 0.8 mL/min to 1.5 mL/min, such as 0.8 mL/min, 0.9 mL/min, 1.0 mL/min, 1.1 mL/min, 1.2 mL/min, 1.3 mL/min, 1.4 mL/min, or 1.5 mL/min. Preferably, the flow rate is 0.8 mL/min.

Preferably, the chromatographic column has a specification of 4.6×150 mm, 3 μm.

Preferably, the chromatographic column is selected from one of a Waters chromatographic column, an Agilent chromatographic column, a Thermo chromatographic column, a Phenomenex chromatographic column, a Shimatsu chromatographic column, or a YMC chromatographic column. In a specific embodiment, the chromatographic column is YMC-Triart C18.

Further, the HPLC adopts gradient elution as follows:
0 min: a volume ratio of the mobile phase A to the mobile phase B is 100:0;
8 min: the volume ratio of the mobile phase A to the mobile phase B is 83:17 to 87:13;
8 min to 13 min: the volume ratio of the mobile phase A to the mobile phase B is maintained at 83:17 to 87:13; and
13 min to 20 min: the volume ratio of the mobile phase A to the mobile phase B is 100:0.

Preferably, the gradient elution is as follows:
0 min: a volume ratio of the mobile phase A to the mobile phase B is 100:0;
8 min: the volume ratio of the mobile phase A to the mobile phase B is maintained at 85:15;
8 min to 13 min: the volume ratio of the mobile phase A to the mobile phase B is maintained at 85:15; and
13 min to 20 min: the volume ratio of the mobile phase A to the mobile phase B is 100:0.

In a specific embodiment, the gradient elution is as follows:

| Time (min) | Mobile phase A (%) | Mobile phase B (%) |
|---|---|---|
| 0 | 100 | 0 |
| 8 | 85 | 15 |
| 13 | 85 | 15 |
| 13.1 | 100 | 0 |
| 20 | 100 | 0 |

Further, in some specific embodiments that require quantification, the method also includes calculating an integral area of a peak.

In a second aspect, the present disclosure provides a use of the method described above in quality control for a germinal peptide preparation.

In some specific embodiments, the use includes: using the method to detect a purity of germinal peptide and analyze impurity species in and/or a purity of a germinal peptide preparation.

In a specific embodiment, the germinal peptide preparation is a germinal peptide eye drop.

The method of the present disclosure is conducted under the following chromatographic conditions: an amino acid/peptide chromatographic column with octadecylsilane-bonded silica gel as a packing (4.6×150 mm, 3 μm); mobile phases: A: ultrapure water (UPW) (0.1% TFA), and B: acetonitrile (0.1% TFA); a detection wavelength: 210 nm; a column temperature: 35° C.; and a flow rate: 0.8 mL/min to 1.5 mL/min. The method of the present disclosure can effectively detect and separate the 3 known impurities (Glu⁷-germinal peptide, D-Leu¹-germinal peptide, and Di-Glu⁵-germinal peptide) in the germinal peptide preparation to eliminate their interference, and shortens a detection time to about 20 min. Thus, with the method of the present disclosure, quality standards are established for germinal peptide.

### BRIEF DESCRIPTION OF THE DRAWINGS

FIG. 1 is a spectrum of germinal peptide detection in the prior art;

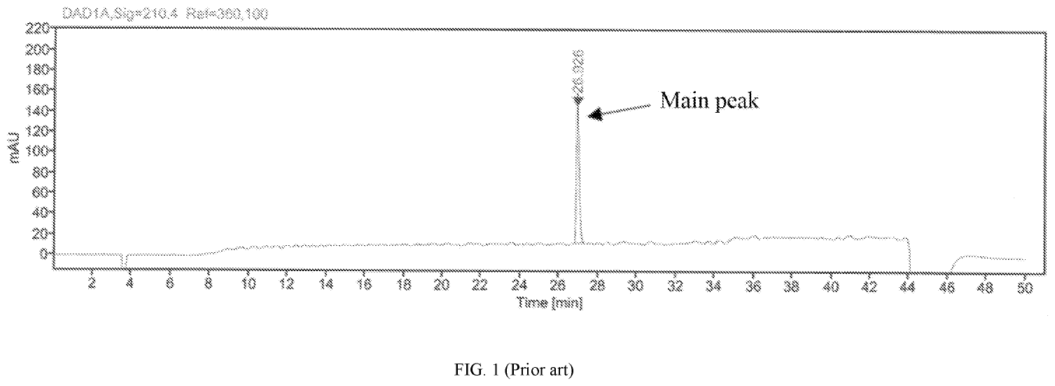

FIG. 2 is a spectrum of API and a related substance in a germinal peptide preparation in the prior art;

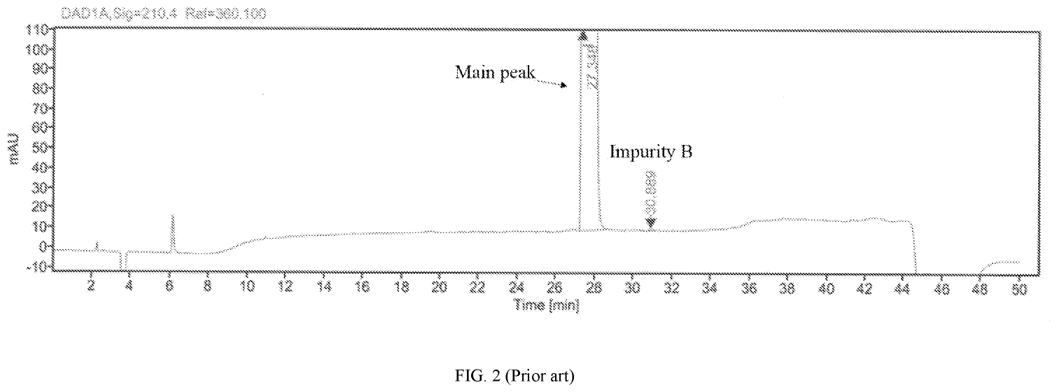

FIG. 3 is a spectrum of API and other related substances in a germinal peptide preparation in the prior art;

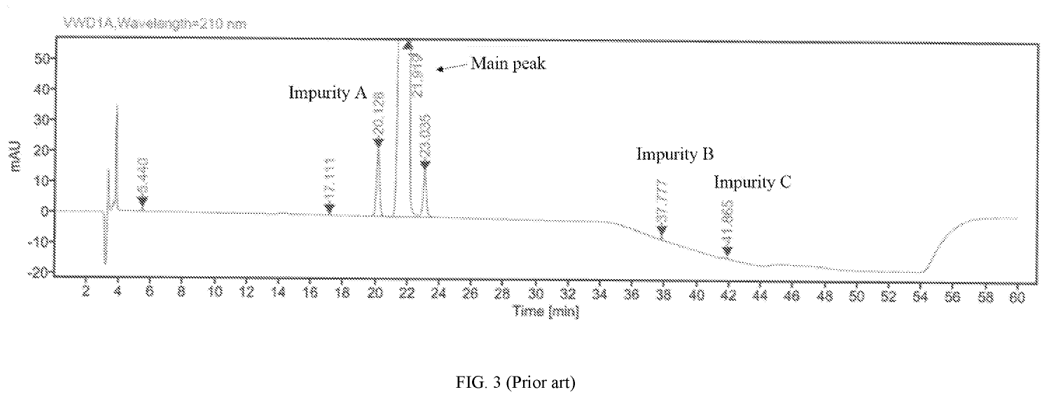

FIG. 4 is a spectrum under optimized conditions in Example 1;

FIG. 5 is a spectrum of isocratic elution in Example 2;

FIG. 6 is a spectrum after an injection volume is reduced in Example 3; and

FIG. 7 is a spectrum after gradient elution is improved in Example 4.

### DETAILED DESCRIPTION OF THE EMBODIMENTS

The present disclosure discloses a method for analysis of related substances in a germinal peptide preparation, and those skilled in the art may appropriately improve process parameters with reference to the content of the present disclosure to implement the method. In particular, it should be noted that all similar substitutions and alterations are apparent to those skilled in the art, and all are deemed to be included in the present disclosure. The method of the present disclosure has been described through preferred embodiments, and those skilled in the art can obviously modify or appropriately change and combine the methods and applications described herein without departing from the content, spirit, and scope of the present disclosure to implement and apply the technology of the present disclosure.

Unless otherwise defined, all scientific and technical terms used in the present disclosure have the same meanings as generally understood by those skilled in the technical field to which the present disclosure belongs.

In the present disclosure, the term “mobile phase” refers to a system that carries a component to be tested and moves forward during a chromatographic process. A mobile phase is in an equilibrium state with a stationary phase.

In order to enable those skilled in the art to well understand the technical solutions of the present disclosure, the present disclosure is further described in detail below in conjunction with specific examples.

In the following examples, germinal peptide is from Zhejiang Peptites Biotech Co., Ltd. and has a batch No. PT2021122101; water is UPW; and other reagents all are analytically-pure reagents.

### Example 1

A germinal peptide eye drop was prepared as follows:
1) About 70% of a prescribed amount of water for injection (WFI) was added to a solution preparation tank with a specified capacity.
2) A prescribed amount of an inorganic salt was added to a stainless steel barrel, about 15% of the prescribed amount of WFI was added to the volumetric flask to obtain a first mixture, and the first mixture was stirred for dissolution; and a prescribed amount of germinal peptide was added to obtain a second mixture, and the second mixture was stirred for dissolution to obtain a clear solution.
3) The clear solution was transferred to a 25 L solution preparation tank, the solution preparation tank originally holding the clear solution was washed 3 times with about 10% of the prescribed amount of WFI, resulting washing solutions were added to the solution preparation tank to obtain a third mixture, and the third mixture was stirred for 5 min to allow thorough mixing.
4) An appropriate amount of hydrochloric acid was taken and slowly added to cooled WFI to prepare a 1 mol/L hydrochloric acid solution for later use.
5) Stirring was started in the solution preparation tank, the prepared hydrochloric acid solution was slowly added under stirring to adjust a pH to 6.5 to 7.5, and a resulting system was further stirred for 5 min.
6) WFI was supplemented to a full prescribed amount, then stirring was started and conducted for 10 min to allow thorough mixing, and a sample was collected for detection.

Octadecylsilane-bonded silica gel was used as a packing for a chromatographic column.

### Chromatographic Conditions:

an instrument: Agilent Infinity II;
a detector: Agilent DAD;
a mobile phase A: a 0.1% TFA aqueous solution;
a mobile phase B: 0.1% TFA-acetonitrile;
a chromatographic column: YMC-Triart C184.6×150 mm, 3 μm;
a column temperature: 35° C.; an injection volume: 100 μL; a detection wavelength: 210 nm; and a flow rate: 0.8 mL/min.

Gradient elution was conducted according to Table 1:

TABLE 1
Elution procedure

| Time (min) | Mobile phase A (%) | Mobile phase B (%) |
|---|---|---|
| 0 | 100 | 0 |
| 8 | 85 | 15 |
| 13 | 85 | 15 |
| 13.1 | 100 | 0 |
| 20 | 100 | 0 |

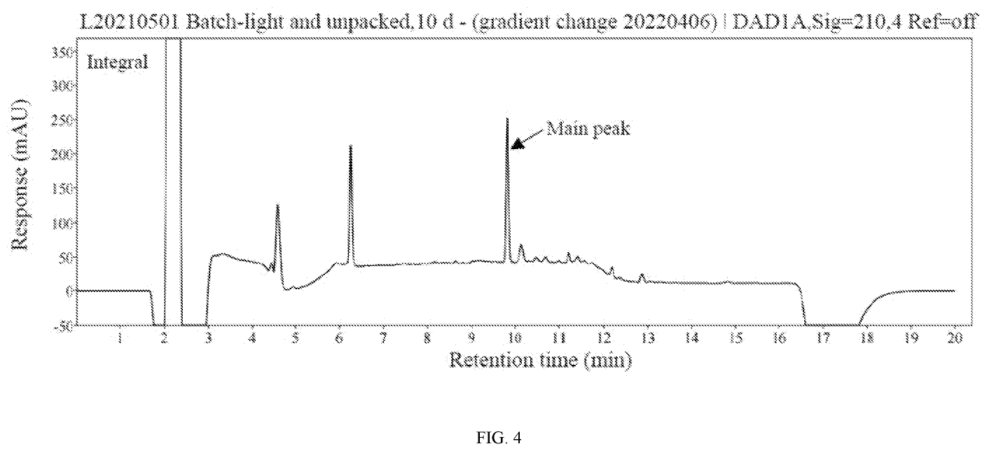

Results are shown in FIG. 4. It can be seen that the method leads to an excellent separation (2.5) of a main peak of germinal peptide from impurity peaks, can improve a peak shape, a tailing (1.1), and a number of theoretical plates (13,000), and greatly shortens a detection time to 20 min, where the main peak has a prominent peak shape, a small tailing factor, and an excellent separation from impurity peaks.

### Example 2: A Chromatographic Column was Changed on the Basis of the Original Chromatographic Conditions, and an Elution Gradient was Adjusted to Isocratic Elution

### Chromatographic Conditions:

a mobile phase A: a 0.1% TFA aqueous solution;
a mobile phase B: 0.1% TFA-acetonitrile;
a chromatographic column: an Agilent Zorbax SB-C18 chromatographic column, 4.6×150 mm, 5 μm;
a column temperature: 35° C.; an injection volume: 100 μL; a wavelength: 210 nm; and a flow rate: 0.8 mL/min.

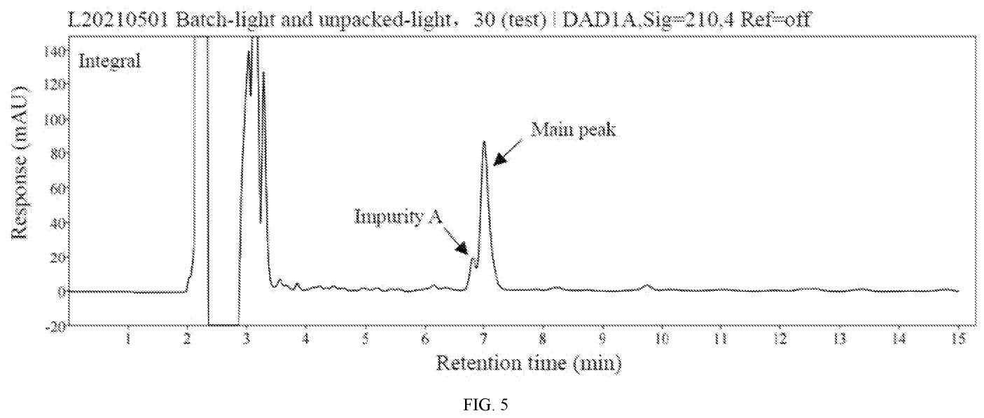

The elution gradient was adjusted to isocratic elution (mobile phase A:B=90:10), the isocratic elution was conducted for 30 min, and an isocratic elution situation for a main peak was observed under an organic phase proportion of 10%. Results are shown in FIG. 5. Under the three gradient conditions, an impurity peak appears at the main peak of germinal peptide, and a separation is small, indicating that, under the isocratic elution conditions, there is a poor separation between the main peak and the impurity peak and the main peak is heavily tailed under a small organic phase proportion.

### Example 3: An Injection Volume was Reduced to 50 μL, and an Elution Gradient was Further Investigated

### Chromatographic Conditions:

a mobile phase A: a 0.1% TFA aqueous solution;
a mobile phase B: 0.1% TFA-acetonitrile;
a chromatographic column: YMC-Triart C184.6×150 mm, 3 μm;
a column temperature: 35° C.; an injection volume: 50 μL; a wavelength: 210 nm; and a flow rate: 0.8 mL/min.

In the first 3 min of elution, a high aqueous phase proportion was adopted for elution to obtain an adjuvant peak with a large polarity, and from 3 min to 15 min, an organic phase proportion of 7% was adopted for elution to obtain a main peak. A peak shape of the main peak and a separation of the main peak from an impurity peak were observed. While ensuring that the appearance of the main peak was not affected by a gradient change, gradient elution was conducted according to Table 2:

TABLE 2
Elution procedure

| Time (min) | Mobile phase A (%) | Mobile phase B (%) |
|---|---|---|
| 0 | 100 | 0 |
| 3 | 98 | 2 |
| 3.1 | 93 | 7 |
| 15 | 93 | 7 |
| 15.1 | 85 | 15 |
| 20 | 85 | 15 |
| 20.1 | 100 | 0 |
| 30 | 100 | 0 |

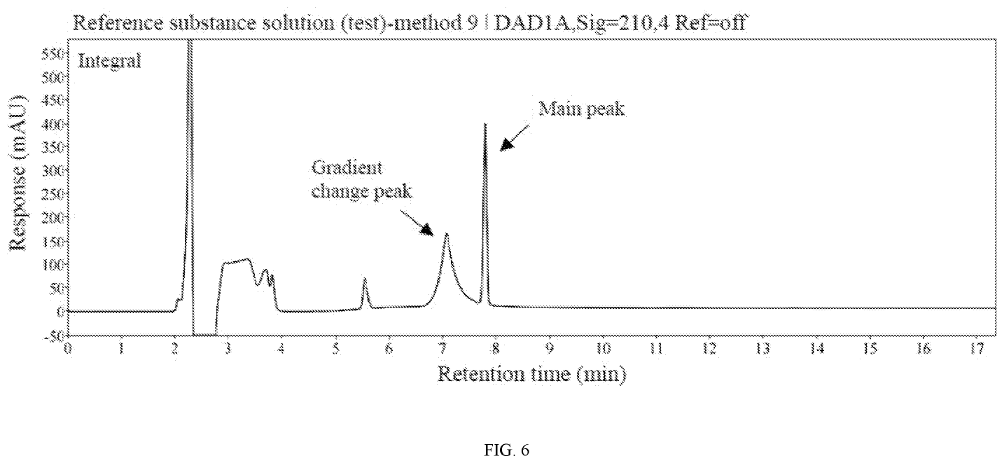

Investigation results are shown in FIG. 6. It can be seen that the main peak is greatly interfered by a gradient change peak.

### Example 4: A Gradient Change Rate was Increased, and a Separation of a Main Peak from an Impurity Peak was Observed

### Chromatographic Conditions:

a mobile phase A: a 0.1% TFA aqueous solution;
a mobile phase B: 0.1% TFA-acetonitrile;
a chromatographic column: YMC-Triart C18, 4.6×150 mm, 3 μm;
a column temperature: 35° C.; an injection volume: 100 μL; a wavelength: 210 nm; and a flow rate: 0.8 mL/min.

Gradient elution was conducted according to Table 3:

TABLE 3
Elution procedure

| Time (min) | Mobile phase A (%) | Mobile phase B (%) |
|---|---|---|
| 0 | 98 | 0 |
| 8 | 85 | 15 |
| 13 | 85 | 15 |
| 13.1 | 100 | 0 |
| 20 | 100 | 0 |

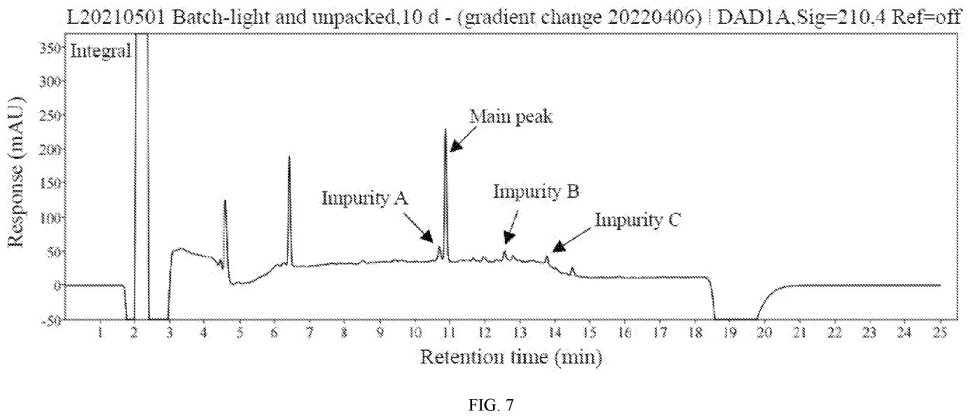

Results are shown in FIG. 7. A separation of a main peak of germinal peptide and an impurity peak in a test sample solution is poor (1.1).

The above are merely preferred examples of the present disclosure and are not intended to limit the present disclosure, and various changes and modifications can be made by those skilled in the art to the present disclosure. Any modifications, equivalent substitutions, improvements, and the like made within the spirit and principle of the present disclosure should be included within the protection scope of the present disclosure.

What is claimed is:

1. A method for an analysis of related substances in a germinal peptide preparation, comprising the following steps:
(1) preparing a sample; and
(2) testing the sample by high-performance liquid chromatography, wherein a chromatographic column with octadecylsilane-bonded silica gel as a packing is adopted for the high-performance liquid chromatography; and mobile phases for the high-performance liquid chromatography comprise a mobile phase A and a mobile phase B, wherein the mobile phase A is a trifluoroacetic acid aqueous solution with a volume ratio of the trifluoroacetic acid to water being 0.05:100 to 0.15:100, and the mobile phase B is trifluoroacetic acid-acetonitrile.

2. The method for the analysis of the related substances in the germinal peptide preparation according to claim 1, wherein a volume ratio of trifluoroacetic acid to acetonitrile in the mobile phase B is 0.05:100 to 0.15:100.

3. The method for the analysis of the related substances in the germinal peptide preparation according to claim 1, wherein a column temperature of the chromatographic column is 30° C. to 40° C.

4. The method for the analysis of the related substances in the germinal peptide preparation according to claim 1, wherein an injection volume for the high-performance liquid chromatography is 90 μL to 110 μL.

5. The method for the analysis of the related substances in the germinal peptide preparation according to claim 1, wherein a detection wavelength for the high-performance liquid chromatography is 210 nm.

6. The method for the analysis of the related substances in the germinal peptide preparation according to claim 1, wherein the chromatographic column is selected from one of a Waters chromatographic column, an Agilent chromatographic column, a Thermo chromatographic column, a Phenomenex chromatographic column, a Shimatsu chromatographic column, or a YMC chromatographic column.

7. The method for the analysis of the related substances in the germinal peptide preparation according to claim 1, wherein the chromatographic column has a specification of 4.6×150 mm, 3 μm.

8. The method for the analysis of the related substances in the germinal peptide preparation according to claim 1, wherein the high-performance liquid chromatography adopts a gradient elution as follows:
0 min: a volume ratio of the mobile phase A to the mobile phase B is 100:0;
8 min: the volume ratio of the mobile phase A to the mobile phase B is 83:17 to 87:13;
8 min to 13 min: the volume ratio of the mobile phase A to the mobile phase B is 83:17 to 87:13; and
13 min to 20 min: the volume ratio of the mobile phase A to the mobile phase Bis 100:0.

9. The method for the analysis of the related substances in the germinal peptide preparation according to claim 1, wherein the high-performance liquid chromatography adopts a gradient elution as follows:
0 min: a volume ratio of the mobile phase A to the mobile phase B is 100:0;
8 min: the volume ratio of the mobile phase A to the mobile phase B is 85:15;
8 min to 13 min: the volume ratio of the mobile phase A to the mobile phase B is 85:15; and
13 min to 20 min: the volume ratio of the mobile phase A to the mobile phase B is 100:0.

10. A method for controlling a quality of a germinal peptide preparation, comprising using the method for the analysis of the related substances in the germinal peptide preparation according to claim 1.

11. The method for controlling the quality of the germinal peptide preparation according to claim 10, wherein in the method for the analysis of the related substances in the germinal peptide preparation, a volume ratio of trifluoroacetic acid to acetonitrile in the mobile phase B is 0.05:100 to 0.15:100.

12. The method for controlling the quality of the germinal peptide preparation according to claim 10, wherein in the method for the analysis of the related substances in the germinal peptide preparation, a column temperature of the chromatographic column is 30° C. to 40° C.

13. The method for controlling the quality of the germinal peptide preparation according to claim 10, wherein in the method for the analysis of the related substances in the germinal peptide preparation, an injection volume for the high-performance liquid chromatography is 90 μL to 110 μL.

14. The method for controlling the quality of the germinal peptide preparation according to claim 10, wherein in the method for the analysis of the related substances in the germinal peptide preparation, a detection wavelength for the high-performance liquid chromatography is 210 nm.

15. The method for controlling the quality of the germinal peptide preparation according to claim 10, wherein in the method for the analysis of the related substances in the germinal peptide preparation, the chromatographic column is selected from one of a Waters chromatographic column, an Agilent chromatographic column, a Thermo chromatographic column, a Phenomenex chromatographic column, a Shimatsu chromatographic column, or a YMC chromatographic column.

16. The method for controlling the quality of the germinal peptide preparation according to claim 10, wherein in the method for the analysis of the related substances in the germinal peptide preparation, the chromatographic column has a specification of 4.6×150 mm, 3 μm.

17. The method for controlling the quality of the germinal peptide preparation according to claim 10, wherein in the method for the analysis of the related substances in the germinal peptide preparation, the high-performance liquid chromatography adopts a gradient elution as follows:
0 min: a volume ratio of the mobile phase A to the mobile phase B is 100:0;
8 min: the volume ratio of the mobile phase A to the mobile phase B is 83:17 to 87:13;
8 min to 13 min: the volume ratio of the mobile phase A to the mobile phase B is 83:17 to 87:13; and
13 min to 20 min: the volume ratio of the mobile phase A to the mobile phase B is 100:0.

18. The method for controlling the quality of the germinal peptide preparation according to claim 10, wherein in the method for the analysis of the related substances in the germinal peptide preparation, the high-performance liquid chromatography adopts a gradient elution as follows:
0 min: a volume ratio of the mobile phase A to the mobile phase B is 100:0;
8 min: the volume ratio of the mobile phase A to the mobile phase B is 85:15;
8 min to 13 min: the volume ratio of the mobile phase A to the mobile phase B is 85:15; and
13 min to 20 min: the volume ratio of the mobile phase A to the mobile phase B is 100:0.
